(19) United States
(12) Patent Application Publication
(10) Pub. No.: US 2011/0208174 A1
(43) Pub. Date: Aug. 25, 2011

(54) CRYOCLAMP AND METHOD OF USE

(75) Inventor: John M. Baust, Owego, NY (US)

(73) Assignee: CPSI BIOTECH, Owego, NY (US)

(21) Appl. No.: 13/027,856

(22) Filed: Feb. 15, 2011

Related U.S. Application Data
(60) Provisional application No. 61/307,170, filed on Feb. 23, 2010.

(51) Int. Cl. A61B 18/02

(52) U.S. Cl. 606/21

(57) ABSTRACT

A cryogenic medical device is disclosed for use in minimally invasive surgical procedures. Various configurations of cryoprobes are designed in combination with a clamp to form a cryoclamp for the treatment of damaged, diseased, cancerous or other unwanted tissues. The device is an integrated cryoablation probe with a hinged clamp that allows for single entry into the chest cavity through a thorascopic port, by surgical or other means. The integrated cryoablation probe allows for the clamping of tissue as well as freezing with a single device. The clamp acts as an outer sheath so that when closed, directional freezing of the cryoprobe is achieved on the opposing probe surface away from the clamp or on an internal surface that is between the clamp. The cryoclamp may be a removable attachment or integrated into the unitary device.

### RELATED APPLICATIONS

The present application claims priority to U.S. Provisional Patent Application No. 61/307,170 filed on Feb. 23, 2010 and titled A Cryoclamp and Method of Use, which is incorporated herein.

### FIELD OF THE INVENTION

The present invention relates generally to the medical treatment technology field and, in particular, to a device for use in cryo-therapeutic procedures.

### BACKGROUND OF THE INVENTION

Cryotherapy is an effective yet minimally invasive alternative to surgery, radiofrequency (RF) and high-intensity focused ultrasound (HIFU). In this minimally invasive procedure, the destructive forces of freezing are utilized to ablate unwanted tissue in a way that decreases hospitalization time, reduces postoperative morbidity, decreases return interval to daily activities, and reduces overall treatment cost compared to conventional treatments.

Cryosurgery has been shown to be an effective therapy for a wide range of tumor ablation as well as its use to treat atrial fibrillation. Since the early 1960s, treatment of tumors and unwanted tissue has developed around freezing techniques and new instrumentation and imaging techniques to control the procedure. As a result, the complications of cryoablation have been reduced and the efficacy of the technique has increased.

Current atrial fibrillation surgical cryoablation uses two separate devices, a probe and a clamp, to freeze pulmonary veins and atrial appendages. The clamp and probe are bulky, ineffective and difficult to maneuver. Clamping of the structure affects the proper freezing of the tissue. In addition, use of these items has been invasive, thus requiring incisions into the chest to clamp veins and tissue; and then another instrument is used for the freezing.

There exists a need to avoid injury to important adjacent structures while minimizing the invasiveness and aggressiveness of surgery. Improvements in minimizing unwanted post-operative complications will reduce the number of invasive probes into the body during surgery, while achieving the same or better efficacy in treatment.

The novelty of the present invention utilizes an integral device to effectively perform multiple functions. The device will include a means for clamping and securing veins and atrial appendages, or other tissue, while further improving the treatment functionality. The invention will desirably clamp and cryotreat the designated tissue.

Due to its effectiveness as a minimally invasive treatment, the invention will not only facilitate the eradication of tissue, but also decrease hospitalization time, limit postoperative morbidities, shorten return to daily functions and work, and further reduce the overall treatment cost. Desirably, these improvements to the cryo-therapeutic procedure will advantageously provide better health treatment options and eliminate unnecessary health effects and time delays that negatively impact healthcare overall.

### SUMMARY OF THE INVENTION

An embodiment of the invention is a cryoclamp, an integrated cryoablation probe with a hinged clamp to allow for single entry into the chest through a thorascopic port, other surgical means, or any means of access to any area of a body. The clamp allows for clamping of tissue and freezing with a single device. Further, the clamp acts as an insulative outer sheath so that when closed and clamped against the tissue, freezing of the cryoprobe is achieved on an opposite or opposing probe surface away from the internal grip of the clamp. The freeze zone may be on a surface internal to the clamp as varied by the method of implementation.

In one embodiment of the invention, a medical instrument comprises: a longitudinal body having at least one treatment surface; an articulating joint attached to at least a portion of the longitudinal body; and an extension having a proximal end and a distal end; the extension aligned with the longitudinal body and attached to the articulating joint at a proximal end; wherein the articulating joint reversibly adjusts to an open and closed position to form a clamp for securing a tissue structure between the longitudinal body and the extension. The medical instrument has at least one treatment surface to create a linear ablation. Such ablation can include cryogenic treatment, radiofrequency ablation (RF), high-intensity focused ultrasound (HIFU), laser ablation, or other means of ablation.

One embodiment utilizes a cryogenic treatment to create a directional freeze zone along a linear path. In positioning the clamp, the treatment surface may be positioned between the longitudinal body and the extension, or on an opposing surface outside of the clamp. One or more probes or catheters may be implemented, including versatility in deflection and flexible configurations. In one aspect, the longitudinal body deflects at the articulating joint, alone or in combination with the extension to form a diverted probe or catheter.

The invention also encompasses a method of using the medical instrument described, the method comprising the steps of: preparing the medical instrument for contact with a tissue internal to a mammalian body; positioning the tissue in a first position between the longitudinal body and the extension; securing the tissue into a clamped position; activating a first procedure, the first procedure being an ablative treatment; ceasing the ablative treatment; and removing the medical instrument from the tissue.

In addition, one embodiment of the invention is a medical instrument defined as a cryoinstrument comprising: a longitudinal body having at least one treatment surface which creates a directional freeze zone; an articulating joint attached to at least a portion of the longitudinal body; and an extension having a proximal end and a distal end, the extension aligned with the longitudinal body and attached to the articulating joint at a proximal end; wherein the articulating joint reversibly adjusts to an open position and a closed position to form a clamp for securing a tissue structure between the longitudinal body and the extension. In one aspect, the articulating joint is integral with the longitudinal body such that the clamp can be utilized with any probe or catheter. Thus the longitudinal body and/or the extension can be configured as a probe or catheter.

In another aspect, the articulating joint adjusts along the longitudinal body to accommodate any size and shape of extension or additional component to form the clamp. The clamp, its extension or its components, including the articulating joint can be attachable components removably positioned with said longitudinal body. The extension or various features of the probe or catheter are reversibly secured to the tissue structure for easy on and easy off clamping. The longitudinal body of the cryoinstrument comprises a freeze segment in the range of about 0.5 cm to 15 cm or greater; its diameter being in the range of about 1.5 mm to 10 mm.

Where the medical instrument is a cryoinstrument, a cryogenic fluid medium is used for cooling the system, the cryogenic fluid medium comprising any of the following, alone or in combination, including: nitrogen, carbon dioxide, argon, nitrous oxide, propane, and other desirable cryogenic fluids. In one embodiment of the invention, the cryogenic fluid medium utilized for the probe and/or catheter cooling is supercritical nitrogen.

In one aspect, the probe or the catheter includes features for operability and measurement, including mechanisms having computerized or remote control, motorized components, pull-wires, hydraulics, pneumatics, and sensors for remote operation. Other features monitor or control temperature, pressure, positioning, and electrophysiology measurements.

Various embodiments of the invention allow the clamp to be adjusted and implemented for a second procedure at the same tissue site or a second tissue site. Thus, modifications deemed obvious may be integrated and combined in various sizes, shapes, and configurations.

### BRIEF DESCRIPTION OF THE DRAWINGS

The invention is best understood from the following detailed description when read with the accompanying drawing figures. It is emphasized that the various features are not necessarily drawn to scale. In fact, the dimensions may be arbitrarily increased or decreased for clarity of discussion.

FIG. 1: An illustrative embodiment of the device in an open configuration.

FIG. 2: An illustrative embodiment of the device in a closed position.

FIG. 3: An illustrative embodiment of the present invention in a closed position.

FIG. 4: A depiction of an embodiment of the integrated clamp of the present invention.

FIG. 5: An embodiment of the medical device having more than one treatment surface integrated with the clamp.

### DETAILED DESCRIPTION

In the following detailed description, for purposes of explanation and not limitation, exemplary embodiments disclosing specific details are set forth in order to provide a thorough understanding of the present invention. However, it will be apparent to one having ordinary skill in the art that the present invention may be practiced in other embodiments that depart from the specific details disclosed herein. In other instances, detailed descriptions of well-known devices and methods may be omitted so as not to obscure the description of the present invention.

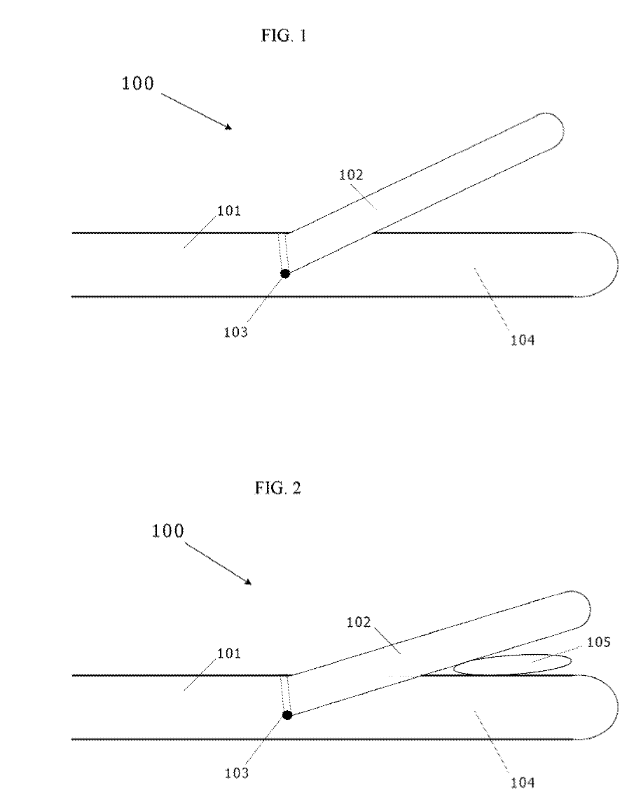

A side view of a cryoclamp in accordance with one embodiment of the present invention is illustrated in FIG. 1. The integrated device 100 has a longitudinal body 101 which includes the mechanical aspects of a cryoprobe 101. A first arm 102 attaches at an integration, or articulation point 103 to allow the first arm 102 to function as a clamp 102 and close upon the extended body or probe extension 104. In this depiction of the device 100, the clamp 102 is in an open configuration which would allow the placement of tissue in the open space between the clamp 102 and the cryoprobe 104.

In one embodiment, the clamp component is mechanically engineered for manual operation. Another embodiment utilizes a cabling material to provide adjustable forces and tension in clamping the tissue. A pressurized pneumatic cylinder or hydraulic device would also be capable of controlling the operation of the clamp (e.g. from an open to closed position and vice versa). In other aspects, the mechanical operation of the clamp may include motorized components, pull-wires, hydraulics, and pneumatics. The clamp may also have a controllable articulation that can be achieved by a micro-sized motor. Any manual or computerized remote control operation of the device is possible. In one aspect, the remote control operations are wireless controls including various sensors for monitoring and controlling temperature, pressure, positioning of the clamp, and electrophysiology measurements. In another aspect, the remote control operation is wired to the handheld device or directly to the cryosystem, such that all control mechanisms would originate from a central location (whether that be at the cryosource, within the handheld instrument itself, or within a remote control separate from the medical device).

In FIG. 2, an illustrative embodiment of the device 100 is depicted in a closed position with tissue 105 clamped in the space between clamp 102 and cryoprobe extension 104 of the body 101. In one embodiment of the device 100, the clamp 102 is an integral component of the body 101 to form a unitary cryoclamp 100. In another embodiment, the device 100 may include an attachable or attached fixture which slides onto or affixes to existing probes or catheters. The slide-on clamp could comprise a ring (e.g. rigid or flexible material composition) or attachment unit that would have complementary fit with a separate probe or catheter device. In one aspect, the attachable clamp device transforms a standard probe into a cryoclamp. In another aspect, the attachable clamp device is moldable or adaptable and configured for reversible attachment onto any medical or surgical instrument. For exemplary purposes, and not limitation, the clamp may attach at a first articulation point 103 and be removed and/or reattached at a second articulation point (not illustrated) anywhere along the longitudinal body 101. Such features can easily be modified and adjusted based on the instrument, equipment, or other devices utilized. Multiple attachments and clamps can thus be configured with the use or multiple hinge points.

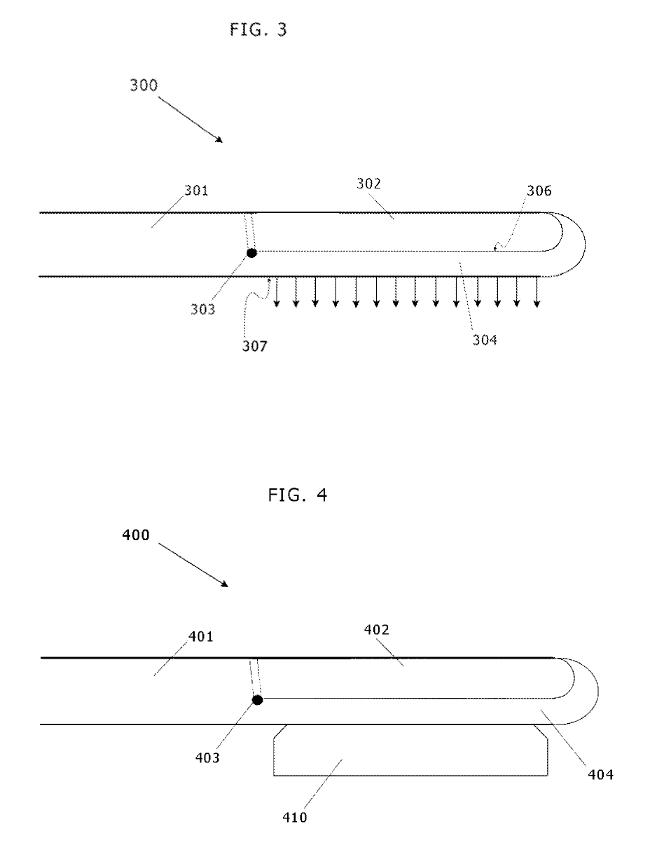

FIG. 3 illustrates a closed cryoclamp 300 comprising a body 301 which utilizes the probe configuration or extension 304. A hinged articulating joint 303 allows the clamp 302 to close upon an inner clamped surface 306 of the probe 304. An outer [unclamped] surface 307 of the probe 304 directs the freeze temperature to an outer non-clamped tissue, uni-directionally treating tissue away from the clamp 302. In one aspect, the inner surface 306 insulates and protects the clamped tissue (e.g. tissue 105 in FIG. 2) from the extreme cold temperatures. In another aspect, the probe 304 can generate multi-directional cryotreatments, from various external surfaces 307 of the probe 304, while excluding treatment near the inner surface 306 of the probe. In yet another aspect, the treatment surface may be the inner surface while the external surface is an insulative barrier.

FIG. 4 illustrates the treatment of tissue structure 410 in an embodiment of the present invention. The body 401 comprises a probe extension 404 connected to a clamp 402 at an integration point, or hinge 403. The cryoclamp device 400 attaches to a vessel or other tissue structure (not illustrated) to secure and/or stabilize the device to prepare for treatment. The designated tissue structure 410 can thereby be treated via cryo-procedures without damaging the clamped tissue 405.

In one embodiment, the probe/catheter extension 404 is a rigid structure. In another embodiment, the probe extension 404 is a flexible tip. Also, sensors along and adjacent to the probe may be positioned on one or more surfaces for the electrical monitoring of the heart or even for temperature monitoring. In other aspects, any number or type of sensors may complement functionality of the probe.

In addition, the probe extension 404 may also incorporate a heating element for warming the device post-treatment. Various aspects of a heating/warming system would include electrical components and/or material compositions compatible with the use of various cryogens and the use of warmer gases.

In addition, the control of the device can be positioned as a trigger control of a hand-held device, remote from a cryogen generator or system. The trigger may have automatic or manual functionality, having a push button control, pull mechanism, or operate as any mechanical trigger. Further, in another aspect, the cryoclamp device 400 and cryogenic generator may be a unitary integral device, handheld, and utilized in a procedure similar to the cryoinject model (e.g. a smaller scale cryogun device separate from the larger and less transportable cryogenic console and attached cryoprobe design).

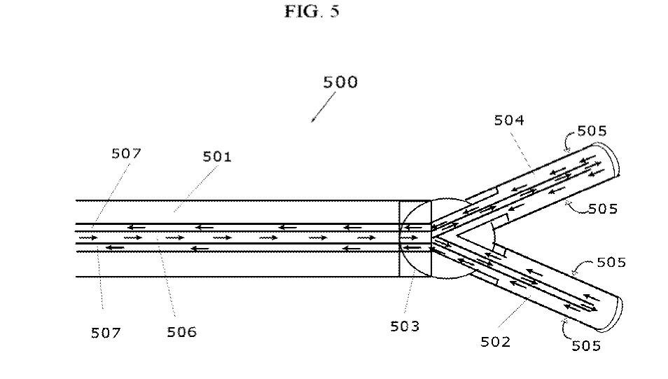

One embodiment, as depicted in FIG. 5, utilizes cryogenic treatment protocols to perform a linear ablation. Here, a longitudinal body 501 integrates a first arm 502 and a second arm 504, each positioned about an articulation joint 503 to form a diverted probe or catheter 500. The first arm 502 and the second arm 504 have deflection capabilities to rotate about the longitudinal axis. The internal supply line 506 supplies the first arm 502 and the second arm 504 with a cryogenic fluid, such as supercritical nitrogen. The return lines 507 deliver the recovered fluid back to the dewar (not depicted) of the closed system. In one aspect, the probes are rigid. In another aspect, the probes may be composed of flexible materials, such as in the configuration of a flexible catheter. A directional freeze zone is created along linear surfaces 505 of the first arm 502 and the second arm 504. While the directional freeze zones illustrated here are between the two probes and on an opposing side of the clamp, the freeze zone may be individual and uni-directional from any surface of the arms 502 and 504 (See unidirectional freeze zone in FIG. 3, as indicated by the arrows. In yet another aspect, the longitudinal body 501 is flexible.

In one embodiment, the device of the invention could be comprised of materials compatible and desirable for use in the medical field. For exemplary purposes, and not limitation, such materials could include metals: stainless steel, copper, gold, aluminum, and tungsten may be of choice. Aluminum may be desirable because it is light weight, inexpensive, easy to machine, biocompatible, and nonmagnetic for MRI use. Other metals, plastics/polymers, and various compositions thereof, however, may be integrated in the material composition to fully realize the various potential applications for utilizing the device. Optical components and/or monitoring sensors may also be desirable to provide for visualization and automatic functioning of the device.

The embodiments of the present invention may be modified to take the shape and have dimensions of any device or apparatus currently used in the industry. Specifically, probe structures utilized to date in cryotherapy or alternative treatment therapy probes, such as those used in radiofrequency treatment, may be modified to include an integration point and clamp attachment. The clamp is compatible with any fluid cryogen system (i.e. gas, liquid, critical or supercritical fluid) at any temperature or pressure, including supercritical nitrogen systems. The clamp may be utilized with any type of cryoprobe, rigid or flexible, including but not limited to surgical probes and catheters. The modified devices and systems which include the integrated clamp design would therefore allow for improved cryogenic or radiofrequency treatment options. Further, any number or combination of arms or clamps may be integrated in combination with the components of the above device. The device and/or system may take many forms and be of any size, shape, or dimension. Any number of sensors or control mechanisms may also be utilized to facilitate operation of the device/system.

For exemplary purposes, and not limitation, the cryoclamp may be a miniaturized version and compact so as to slide through a minute incision. In another aspect, the device may include a locking mechanism while the clamp is in the closed (or open) position. The locking mechanism would ensure that the clamp remains in closed position during the entry and removal from the incision; and then controllably release to clamp and secure the desired tissue. The locking mechanism also serves as a safety feature in precisely locating and securing the desired tissue, whereby sensors therein would add an additional feature to ensure adjacent tissue is not adversely affected.

As presented, the multiple embodiments of the present invention offer several improvements over standard medical devices currently used in the cryogenic industry. The improved cryogenic medical devices disclosed herein remarkably enhance the utilization of a cryoprobe for the freezing of targeted tissue. The present invention provides cost savings in the integrated structure, while reducing the invasiveness of treatment. The previously unforeseen benefits have been realized and conveniently offer advantages for the treatment of multiple disease states. In addition, the improvements enable construction of the device as designed to enable easy handling, storage, and accessibility.

As exemplified, the device may include any cryoprobe or radiofrequency probe with the capacity to integrally incorporate any combination of the disclosed integrated structure(s). The invention being thus described, it would be obvious that the same may be varied in many ways by one of ordinary skill in the art having had the benefit of the present disclosure. Such variations are not regarded as a departure from the spirit and scope of the invention, and such modifications as would be obvious to one skilled in the art are intended to be included within the scope of the following claims and their legal equivalents.

1. A cryoinstrument comprising:
a longitudinal body having at least one treatment surface which creates a directional freeze zone;
an articulating joint attached to at least a portion of said longitudinal body; and
an extension having a proximal end and a distal end; said extension aligned with said longitudinal body and attached to said articulating joint at a proximal end;
wherein said articulating joint reversibly adjusts to an open position and a closed position to form a clamp for securing a tissue structure between said longitudinal body and said extension.

2. The cryoinstrument of claim 1, wherein said articulating joint is integral with said longitudinal body.

3. The cryoinstrument of claim 1, wherein said longitudinal body is a probe or a catheter.

4. The cryoinstrument of claim 1, wherein said extension is a probe or catheter.

5. The cryoinstrument of claim 3, wherein said extension is an insulator.

6. The cryoinstrument of claim 1, further comprising a plurality of articulating joints for providing a range of motion of said clamp.

7. The cryoinstrument of claim 1, wherein said articulating joint adjusts along said longitudinal body.

8. The cryoinstrument of claim 1, wherein said articulating joint is an attachable component removably positioned with said longitudinal body.

9. The cryoinstrument of claim 1, wherein said extension is removable.

10. The cryoinstrument of claim 1, wherein said extension is reversibly secured to said tissue structure.

11. The cryoinstrument of claim 1, wherein said longitudinal body comprises a freeze segment in the range of about 0.5 cm to 15 cm or greater.

12. The cryoinstrument of claim 1, wherein said longitudinal body has a diameter in the range of about 1.5 mm to 10 mm.

13. The cryoinstrument of claim 3, wherein said probe or said catheter utilizes a cryogenic fluid medium for cooling.

14. The cryoinstrument of claim 13, wherein said cryogenic fluid medium comprises any of the following including:
nitrogen;
carbon dioxide;
argon;
nitrous oxide;
propane; and
any combination thereof.

15. The cryoinstrument of claim 14, wherein said cryogenic fluid medium is supercritical nitrogen.

16. The cryoinstrument of claim 3, wherein said clamp includes features for operability and measurement, including mechanisms having computerized or remote control, motorized components, pull-wires, hydrolics, pneumatics, and sensors for remote operation.

17. The cryoinstrument of claim 16, wherein said features monitor or control temperature, pressure, positioning, and electrophysiology measurements.

18. A medical instrument comprising:
a longitudinal body having at least one treatment surface;
an articulating joint attached to at least a portion of said longitudinal body; and
an extension having a proximal end and a distal end; said extension aligned with said longitudinal body and attached to said articulating joint at a proximal end;
wherein said articulating joint reversibly adjusts to an open and closed position to form a clamp for securing a tissue structure between said longitudinal body and said extension.

19. The medical instrument of claim 18, wherein said at least one treatment surface creates a linear ablation.

20. The medical instrument of claim 19, wherein said at least one treatment surface utilizes a cryogenic treatment or a radiofrequency ablation.

21. The medical instrument of claim 20, wherein said treatment surface utilizes a cryogenic treatment and creates a directional freeze zone.

22. The medical instrument of claim 18, wherein said at least one treatment surface is positioned between said longitudinal body and said extension.

23. The medical instrument of claim 18, wherein said at least one treatment surface is positioned on an opposing side of said clamp.

24. The medical instrument of claim 18, wherein said longitudinal body comprises one or more probes or catheters.

25. The medical instrument of claim 24, wherein said longitudinal body deflects at said articulating joint, alone or in combination with said extension, to form a diverted probe or catheter.

26. The medical instrument of claim 24, wherein said one or more probes or catheters create a linear freeze zone, alone or in combination.

27. A method of using said medical instrument of claim 18, said method comprising the steps of:
preparing said medical instrument for contact with a tissue internal to a mammalian body;
positioning said tissue in a first position between said longitudinal body and said extension;
securing said tissue into a clamped position;
activating a first procedure, said first procedure being an ablative treatment;
ceasing said ablative treatment; and
removing said medical instrument from said tissue.

28. The method of claim 27, wherein said ablative treatment comprises cryoablation.

29. The method of claim 27, wherein said ablative treatment comprises radiofrequency (RF), high-intensity focused ultrasound (HIFU), laser ablation or other ablative therapies.

30. The method of claim 27, further comprising a step of adjusting said medical instrument to a second position for a second procedure.

31. The method of claim 28, wherein said step of activating, said cryoablation utilizes a system having pressures in the range of about 22 psi to about 6000 psi.

32. The method of claim 31, wherein said pressures are in the range of about 500 psi to about 1200 psi.

33. The method of claim 27, wherein said cryoablation utilizes a probe or catheter.
